(19) United States
(12) Patent Application Publication
(10) Pub. No.: US 2022/0080016 A1
(43) Pub. Date: Mar. 17, 2022

(54) Antioxidant, Antibacterial Or Anti-Inflammatory Pharmaceutical Composition Containing Scutellaria Baicalensis Extract And Magnolia Officinalis Extract As Active Ingredients

(71) Applicant: Genogen Co.,Ltd., Cheongju-si (KR)

(72) Inventor: Miyoung Lee, Cheongju-si (KR)

(73) Assignee: Genogen Co.,Ltd., Cheongju-si (KR)

(21) Appl. No.: 17/017,876

(22) Filed: Sep. 11, 2020

(51) Int. Cl. A61K 36/575; A61K 36/539; A61P 15/02; A61P 31/10

(52) U.S. Cl. CPC A61K 36/575; A61K 36/539; A61K 2236/333; A61P 31/10; A61P 15/02

(57) ABSTRACT

Disclosed is an antioxidant, antibacterial or anti-inflammatory pharmaceutical composition containing a Scutellaria baicalensis extract and a Magnolia officinalis extract as active ingredients. A pharmaceutical composition containing a mixture of a Scutellaria baicalensis extract and a Magnolia officinalis extract in a specific mixing ratio as an active ingredient has an antioxidant, antibacterial, or anti-inflammatory effect. Specifically, the composition exhibits an antibacterial effect against at least one selected from a group consisting of E. coli, P. aeruginosa, P. acnes, S. aureus, S. epidermidis, B. substilis, C. albicans, and S. cerevisiae. Therefore, the composition has a prophylactic, therapeutic or ameliorating effect of vulvovaginal candidiasis caused by C. albicans.

### TECHNICAL FIELD

The present disclosure relates to an antioxidant, antibacterial or anti-inflammatory pharmaceutical composition containing a Scutellaria baicalensis extract and a magnolia officinalis extract as active ingredients.

### BACKGROUND

Plants have provided a rich and vital source of compounds for the development of medicines. Over the past decades, medicinal plants have been a valuable source of therapeutic agents, and today still many therapeutic drugs are plant-derived natural products or derivatives thereof. Among the several small molecules derived from plants, polyphenols are the most abundant antioxidants in our diets and have been shown to play an important role in the prevention of several diseases. Polyphenol compounds such as pyrogallol remove kainic acid (KA)-induced neuronal damage through oxidative stress inhibition. A wide variety of plant-derived phenolic substances have also been reported to possess distinct antioxidant and anti-inflammatory activity that contribute to their therapeutic function.

A rich source of such phenolic compounds is Scutellaria baicalensis as a flowering plant species of the Lamiaceae family. It is indigenous to several East Asian countries and the Russian Federation and is cultivated in many European countries. It is used as a traditional Chinese medicine for diarrhea, dysentery, hypertension, hemorrhage, insomnia, inflammation, and respiratory infections.

Magnolia trees, on the other hand, are mainly distributed in East and Southeast Asia. The bark of Magnolia officinalis has been traditionally used in Chinese and Japanese medicines for the treatment of anxiety, asthma, depression, gastrointestinal disorders, headaches, etc.

Although these plants have marked therapeutic effects, they also have several adverse effects including cytotoxicity, genotoxicity, carcinogenicity, developmental toxicity, and the like. To overcome these problems, researchers have put a lot of effort.

Factors causing inflammation by causing abnormalities in the body's immune system may be largely divided into bacteria, viruses, and fungi. Examples of bacteria include lactic acid bacteria, E. coli related to food poisoning, O-157 bacteria, Helicobacter pylori, and the like. When disease-related bacteria are infected in the body, they spread rapidly in the body and are likely to be transmitted to air, water, food, etc. Fungi usually include spore-type fungi floating alone, and mycelium-type fungi that are attached onto dust or water droplets and scatter in all directions. Fungi that freely float in the air may cause bronchial asthma or allergic diseases.

Further, candidiasis is a disease that is infected by a fungus called Candida albicans (C. albicans) and is called candida vaginitis or candidiasis. Candida albicans is a fungus that normally exists in humans, but may cause infection when the body's immunity is lowered. Candidiasis is common in infants and the elderly, users of immunosuppressants, cancer patients receiving chemotherapy, and patients with acquired immunodeficiency syndrome. Treatment of candidiasis is not difficult. However, because resistance of Candida albicans to antibiotics commonly prescribed in hospitals gradually increases, the antibiotics alone may not treat the candidiasis effectively. The bacteria and fungi may be directly killed using fungicides and antibiotics, but they may have side effects such as bacterial resistance. Further, side effects including cytotoxicity, genotoxicity, developmental toxicity and carcinogenicity should not be overlooked. Therefore, in order to overcome the above-mentioned side effect problem, many researchers are looking for solutions by combining various mixtures or combinations of both chemical and herbal medicines.

### SUMMARY

The present disclosure has been made in an effort to provide an antioxidant, antibacterial or anti-inflammatory pharmaceutical composition containing a Scutellaria baicalensis extract and a Magnolia officinalis extract in a specific ratio based on parts by weight, in particular, provide an antioxidant, antibacterial, or anti-inflammatory pharmaceutical composition having an effect of preventing, treating or ameliorating vulvovaginal candidiasis.

In addition, the technical purpose to be achieved by the present disclosure is not limited to the technical purpose as mentioned above. Other technical purposes not mentioned may be clearly obvious to those of ordinary skill in the technical field to which the present disclosure belongs from the following descriptions.

An example of the present disclosure provides an antioxidant, antibacterial or anti-inflammatory pharmaceutical composition containing a Scutellaria baicalensis extract and a Magnolia officinalis extract as active ingredients.

In one implementation, the composition contains the Scutellaria baicalensis extract and the Magnolia officinalis extract in a ratio of 4 to 6:1 based on parts by weight.

In one implementation, the Scutellaria baicalensis extract is extracted using water for 1 to 3 hours at 70 to 90° C.

In one implementation, the Magnolia officinalis extract is extracted using 50% ethanol for 1 to 3 hours at 50 to 70° C.

In one implementation, the Scutellaria baicalensis extract is extracted using Scutellaria baicalensis and a solvent in a ratio of 1:7 to 9 based on parts by weight, and the Magnolia officinalis extract is extracted using Magnolia officinalis and a solvent in a ratio of 1:7 to 9 based on parts by weight.

In one implementation, each of the Scutellaria baicalensis extract and the magnolia officinalis extract is a fraction extract obtained by fractionation with at least one fractionation solvent selected from a group consisting of butanol, acetone, phosphoric acid, hexane, ethanol, and water.

In one implementation, the composition exhibits an antibacterial effect against at least one selected from a group consisting of E. coli, P. aeruginosa, P. acnes, S. aureus, S. epidermidis, B. substilis, C. albicans, and S. cerevisiae.

In one implementation, the composition has a prophylactic, therapeutic or ameliorating effect of vulvovaginal candidiasis.

According to the example of the present disclosure, the pharmaceutical composition according to the present disclosure exhibits less cytotoxicity compared to a case when using the Scutellaria baicalensis extract or the Magnolia officinalis extract as a single extract, and has an antioxidant, antibacterial, or anti-inflammatory effect.

Further, the composition according to the present disclosure exhibits an antibacterial effect against at least one selected from a group consisting of E. coli, P. aeruginosa, P. acnes, S. aureus, S. epidermidis, B. substilis, C. albicans, and S. cerevisiae. The composition may have a prophylactic, therapeutic or ameliorating effect of vulvovaginal candidiasis caused by the C. albicans.

The foregoing summary is illustrative only and is not intended to be in any way limiting. In addition to the illustrative aspects, examples, and features described above, further aspects, examples, and features will become apparent by reference to the drawings and the following detailed description.

### BRIEF DESCRIPTION OF THE DRAWINGS

FIG. 1A shows results of high-performance liquid chromatography on a Scutellaria baicalensis extract according to an example of the present disclosure.

FIG. 1B shows results of high-performance liquid chromatography on a magnolia officinalis extract according to an example of the present disclosure.

FIG. 2 shows results of measuring cytotoxicity and anti-inflammatory effect of each of a Scutellaria baicalensis extract, a Magnolia officinalis extract, and a pharmaceutical composition containing a mixture of a Scutellaria baicalensis extract and a Magnolia officinalis extract in a specific ratio based on parts by weight, according to an example of the present disclosure.

FIG. 3 shows results of measuring antibacterial effect via agar disk analysis of each of a Scutellaria baicalensis extract, a Magnolia officinalis extract, and a pharmaceutical composition containing a mixture of a Scutellaria baicalensis extract and a Magnolia officinalis extract in a specific ratio based on parts by weight, according to an example of the present disclosure.

FIG. 4A through 4D show results of measuring antioxidant effect of each of a Scutellaria baicalensis extract, a Magnolia officinalis extract, and a pharmaceutical composition containing a mixture of a Scutellaria baicalensis extract and a Magnolia officinalis extract in a specific ratio based on parts by weight, according to an example of the present disclosure. FIG. 4A shows the inhibitory activity against FeCl₃-induced lipid peroxidation (TBARS generation). FIG. 4B shows 1,1-diphenyl-2-picrylhydrazyl (DPPH) radical scavenging activity in 30 minutes. FIG. 4C shows Western blot results of Nrf2, HO-1, SOD1 and CAT proteins. FIG. 4D shows the measurement of the band density standardized with GAPDH.

### DETAILED DESCRIPTION

In the following detailed description, reference is made to the accompanying drawing, which forms a part hereof. The illustrative examples described in the detailed description, drawings, and claims are not meant to be limiting. Other examples may be utilized, and other changes may be made, without departing from the spirit or scope of the subject matter presented herein.

Hereinafter, an antioxidant, antibacterial or anti-inflammatory pharmaceutical composition containing a Scutellaria baicalensis extract and a Magnolia officinalis extract as active ingredients according to a specific example of the present disclosure will be described in more detail. The term “anti-inflammatory” as used throughout the specification and claims of the present disclosure should be understood as including ameliorating (reduction of symptom) and treatment of an inflammatory disease, and inhibition or delay of occurrence of the disease.

The anti-inflammatory pharmaceutical composition according to an example of the present disclosure may contain a Scutellaria baicalensis extract and a Magnolia officinalis extract as active ingredients.

Scutellaria baicalensis is a medicinal herb made from a root of Scutellaria baicalensis GEORGE as a perennial herbaceous plant belonging to the Lamiaceae family. Scutellaria baicalensis GEORGE grows in grassland in mountain regions, and has a height of 20 to 60 cm. Several stalks come out and grow as apricots, and have hairs and branches. Leaves are opposite to each other and are lanceolate, and have flat edges. Fruits are fruiting in September and are rounded in a calyx. The root is conical, and the flesh is yellow. In herbal medicine, roots are used for antipyretic, diuretic, diarrhea, cholagogue, and anti-inflammatory. Baicalin as one of the major components of Scutellaria baicalensis is known to have anti-diabetic effects. In the present disclosure, Scutellaria baicalensis may be a Scutellaria baicalensis root.

Magnolia officinalis is a deciduous tree belonging to the Magnoliaceae family, and its origin is divided into Chinese Magnolia officinalis and Japanese Magnolia obovata Thunberg. It has the efficacy of jo-sup-so-dam ( [symbol] ) and ha-gi-je-man ( [symbol] ) as an oriental medicine and treats sup-che-sang-jung ( [symbol] ), wan-bi-to-sa ( [symbol] ), sik-juck-ki-che ( [symbol] ), buk-chang-byun-bi ( [symbol] ), and dam-eum-cheon-hae ( [symbol] ). Components of Magnolia officinalis include essential oils such as alpha, beta, and gamma-eudesmol, magnolol, honokiol, tetrahydromagnolol, magnocurarine, magnoflorine, obovatol, obovataldehyde, and saponin. In one example, in the present disclosure, Magnolia officinalis may be a Magnolia officinalis bark.

According to an example of the present disclosure, the composition may contain a Scutellaria baicalensis extract and a Magnolia officinalis extract in a ratio of 4 to 6:1 based on parts by weight.

Specifically, an extraction method for preparing the extract my include at least one selected from the group consisting of hot water extraction method, cold immersion extraction method, reflux cooling extraction method, solvent extraction method, steam distillation method, ultrasonic extraction method, elution method, and compression method known in the art.

According to an example of the present disclosure, the Scutellaria baicalensis extract may be extracted with water at 70 to 90° C. for 1 to 3 hours, and more specifically at 80° C. for 2 hours. In addition, according to an example of the present disclosure, the Magnolia officinalis extract may be extracted with 50% ethanol for 1 to 3 hours at 50 to 70° C., and more specifically at 60° C. for 2 hours. When extraction is performed under the above temperature and time conditions, it is possible to achieve the maximum extraction of the desired active ingredient.

In one example, during the extraction, each of the Scutellaria baicalensis and Magnolia officinalis extract may be extracted using each of Scutellaria baicalensis and Magnolia officinalis and a corresponding solvent in a ratio of 1:7 to 9 based on parts by weight, specifically, in a ratio of 1:8 based on parts by weight.

In addition, each of a Scutellaria baicalensis extract and a Magnolia officinalis extract according to an example of the present disclosure may be a fraction extract obtained by fractionation with at least one fractionation solvent selected from the group consisting of butanol, acetone, phosphoric acid, hexane, ethanol, and water.

In one example, the pharmaceutical composition prepared according to an example of the present disclosure exhibits little cytotoxicity unlike an individual Scutellaria baicalensis extract or an individual Magnolia officinalis extract. The pharmaceutical composition prepared according to an example of the present disclosure exhibits an antibacterial effect against at least one selected from a group consisting of E. coli, P. aeruginosa, P. acnes, S. aureus, S. epidermidis, B. substilis, C. albicans, and S. cerevisiae.

Thus, the pharmaceutical composition has a prophylactic, therapeutic or ameliorating effect of vulvovaginal candidiasis.

In one example, the pharmaceutical composition according to the present disclosure may additionally contain ingredients commonly used in the pharmaceutical composition, in addition to the extracts as the active ingredient. For example, the composition may further contain conventional adjuvants such as an antioxidant, a stabilizer, a solubilizing agent, vitamins, pigments and flavors, and a carrier.

The pharmaceutical composition according to the present disclosure may be prepared in any formulation commonly prepared in the art. For example, the composition may be formulated as a solution, suspension, emulsion, paste, gel, cream, lotion, powder, soap, oil, powder foundation, emulsion foundation, wax foundation, spray, etc., but is not limited thereto.

In an example, the pharmaceutical composition may be prepared into a formulation of an external preparation for skin. When the composition is prepared into the formulation of the external preparation for skin, the formulation may include a formulation of an essence, cream, pack, cleanser, gel or skin adhesive type, and may include a formulation of a transdermal dosage form such as a lotion, ointment, gel, cream patch or spray.

In addition, in the composition for external application of each formulation, components other than the essential components may be appropriately selected and added by a person skilled in the art without difficulty according to the formulation or purpose of use of other external preparations.

Specifically, when the formulation according to the present disclosure is a paste, cream or gel, a carrier component may include animal oil, vegetable oil, wax, paraffin, starch, tracant, cellulose derivative, polyethylene glycol, silicone, bentonite, silica, talc, zinc oxide, or the like. When the formulation according to the present disclosure is powder or spray, a carrier component may include lactose, talc, silica, aluminum hydroxide, calcium silicate or polyamide powder. In particular, when the formulation according to the present disclosure is spray, the formulation may additionally contain a propellant such as chlorofluorohydrocarbon, propane/butane or dimethyl ether.

When the formulation according to the present disclosure is a solution or emulsion, a solvent, solubilizing agent or emulsifying agent may be used as a carrier component. For example, water, ethanol, isopropanol, ethyl carbonate, ethyl acetate, benzyl alcohol, benzyl benzoate, propylene glycol, 1,3-butyl glycol oil, glycerol aliphatic ester, polyethylene glycol or fatty acid ester of sorbitan may be used as the carrier component.

When the formulation according to the present disclosure is a suspension, a carrier component may include a liquid diluent such as water, ethanol or propylene glycol, suspending agents such as ethoxylated isostearyl alcohol, polyoxyethylene sorbitol ester and polyoxyethylene sorbitan ester, microcrystalline cellulose, aluminum metahydroxide, bentonite, agar or tracant, and the like.

Hereinafter, the present disclosure will be described in detail based on Examples. The following Examples are merely illustrative of the present disclosure, and the scope of the present disclosure is not limited to the following Examples.

### Preparation Example

The Scutellaria baicalensis root (SBR) and the Magnolia officinalis bark (MOB) were collected at Geochang-gun, Gyeongsangnam-do, Korea, in May 2013 and May 2015, respectively, and completely dried under sunlight to obtain the dried plants. The dried Scutellaria baicalensis roots and Magnolia officinalis barks were then separately ground in a laboratory rotor mill pulviresette (Laval Lab Inc., Laval, Canada), and the pulverized products were sterilized by spraying with 70% ethanol, followed by drying at 80° C. for 24 h and storage at 4° C. All extract preparation steps were performed separately for SBR and MOB.

Scutellaria Baicalensis Root (Hereinafter Referred to as SBR) Extract Preparation

Finely ground SBR powder prepared as in the above Preparation Example was extracted with water at 80° C. for 2 h. The ratio of the root powder to water was 1:8 based on parts by weight. The resulting slurries were filtered through Whatman Grade 1 filter paper. This procedure was repeated twice for the residue, and the filtrates were combined.

The extracted solution was then extracted with 1.5% phosphoric acid for 30 min. Removal of the solvent was done under reduced pressure in a rotary evaporator (Rotavapor; BUCHI Labortechnik AG, Flawil, Switzerland) and freeze-dried (Labconco, Kansas City, Mo., USA).

The obtained fraction extract powder was desiccated and stored at 4° C. and used directly in the antioxidant tests. Each fraction extract was dissolved in 10% dimethyl sulfoxide (DMSO), and stock solutions were prepared at high concentrations to ensure that the DMSO concentration did not exceed 0.1% in any of the experiments. Via this process, Scutellaria baicalensis fraction extract was prepared. Table 1 below shows the content of baicalin based on the ethanol content and extraction time.

TABLE 1

| Extraction | Ethanol content | | | | |
|---|---|---|---|---|---|
| time | 0% | 20% | 40% | 50% | 60% |
| 1 h | 13.1% | 11.3% | 10.3% | 10.6% | 10.2% |
| 2 h | 18.2% | 13.2% | 10.5% | 10.3% | 10.3% |
| 3 h | 18.0% | 13.3% | 9.4% | 10.5% | 10.2% |
| 4 h | 17.3% | 13.6% | 9.5% | 10.2% | 10.7% |

Magnolia officinalis Bark (Hereinafter Referred to as MOB) Extract Preparation

Finely ground MOB powder prepared as in the above Preparation Example was extracted with 50% ethanol at 60° C. for 2 h. In this connection, the ratio of Magnolia officinalis bark powder and 50% ethanol was 1:8 based on parts by weight. The filtering step was performed in the same manner as described above for the SBR extract.

Next, the extract was fractionated with hexane for 0.5 hours. After removal of the solvent through evaporation and then the drying, the preparation of the stock solution was performed in the same manner as described above to prepare the Magnolia officinalis bark (MOB) fraction extract. Table 2 below shows the content of magnolol and honokiol based on the ethanol content and extraction time.

TABLE 2

| | Ethanol content | | | |
|---|---|---|---|---|
| Extraction time | 0% | 50% | 70% | 80% |
| 1 h | 12.0% | 12.3% | 11.2% | 9.1% |
| 2 h | 12.2% | 13.4% | 11.0% | 9.2% |
| 3 h | 12.1% | 13.2% | 11.3% | 9.5% |
| 4 h | 12.2% | 13.1% | 11.3% | 9.3% |

### Present Example 1

A water extract of Scutellaria baicalensis root (SBR) with the highest baicalin content and a 50% ethanol extract of Magnolia officinalis bark (MOB) with the highest content of magnolol and honokiol in the above Preparation Example were prepared, and then mixed with each other at a ratio of 5:1 based on parts by weight. A composition (hereinafter, GenoTX-407) containing SBR extract and MOB extract was prepared.

### Comparative Example 1

A composition was prepared in the same manner as in Present Example 1, but the composition contained only the Scutellaria baicalensis root (SBR) extract.

### Comparative Example 2

A composition was prepared in the same way as in Present Example 1, but the composition contained only Magnolia officinalis bark (MOB) extract.

[Experiment 1: High Performance Liquid Chromatography (HPLC) Analysis]

Each sample (200 mg of dried SBR or MOB) was weighed in a volumetric flask, and 50 mL of 10% methanol was added thereto. The solution was then sonicated for 60 min.

After cooling, the solution was filtered using a 0.2 μm syringe filter (PVDF, Whatman plc, Maidstone, UK) and injected into the ACQUITY ultra-high performance liquid chromatography (UHPLC) system (Waters Co., Milford, Mass., USA). The output signal was monitored at a wavelength of 276 nm and processed using Empower 2 software (Waters Inc.).

Chromatographic separation was performed using a Supelco Discovery C18 column (5 μm, 4.6×250 mm; Supleco, Taiwan, ROC). The column and autosampler tray temperatures were maintained at 25° C. The mobile phases A and B were 1% phosphoric acid (v/v) and acetonitrile, respectively. Gradient elution was as follows: 0 to 10 min, 5 to 50% solvent B; 10 to 20 min, 50 to 70% solvent B; and 20 to 50 min 70 to 70% solvent B.

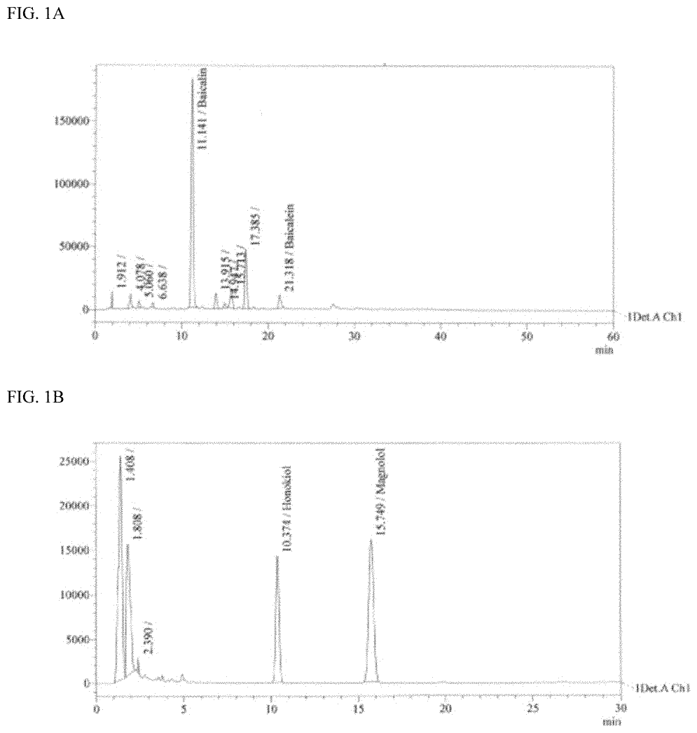

The result of performing the chromatography is shown in FIGS. 1A and 1B. The peak in FIG. 1A represents the baicalin present in the root of Scutellaria baicalensis (SBR), and the peak in FIG. 1B represents the honokiol and magnolol present in the bark of magnolia officinalis (MOB). The total amount of baicalin in the SBR was maximized for 2 hours in 0% ethanol and for 0.5 hours in the 1.5% phosphoric acid fraction. The total amount of magnolol and honokiol in the MOB was maximized for 2 hours in 50% ethanol and 0.5 hours in the hexane fraction. Each polyphenol was identified using HPLC (FIGS. 1A and 1B).

[Experiment 2: MTT Analysis]

Cell Culture

The RAW 264.7 (mouse leukemic monocyte macrophage) cell line and HeLa cells were cultured in Dulbecco's modified Eagle's medium (DMEM; Biowest, Nuaillé, France) containing antibiotics (100 IU/mL penicillin and 100 μg/mL streptomycin) plus 10% heat-inactivated fetal bovine serum (Biowest) at 37° C. in a 5% CO₂/95% air atmosphere. In all experiments, cells were grown until 90% or more confluent and subjected to no more than 20 passages.

Cytotoxicity Assessment

To evaluate the cytotoxicity of Present Example 1 and Comparative Examples 1 to 2, RAW 264.7 cells (1×10⁶ cells/mL) were seeded into each well of a 96-well plate. After a 24 h incubation at 37° C. in a 5% CO₂/95% air atmosphere, the cells were treated with various concentrations (0 to 1000 μg/mL) of Present Example 1 and Comparative Examples 1 to 2, that is, SRB, MOB, and GenoTX-407 and incubated again for 24 h. The cells were then washed twice with fresh medium, after which 10 μl of MTT (5 μg/mL in DMEM) was added thereto, and incubated for an additional 2 h. The medium was discarded, and the formazan formed in the living cells was dissolved in 50 μl of DMSO. The absorbance was measured at 570 nm within 30 min using a microplate reader. The experiments were performed in triplicate, and mean and standard deviation values were calculated.

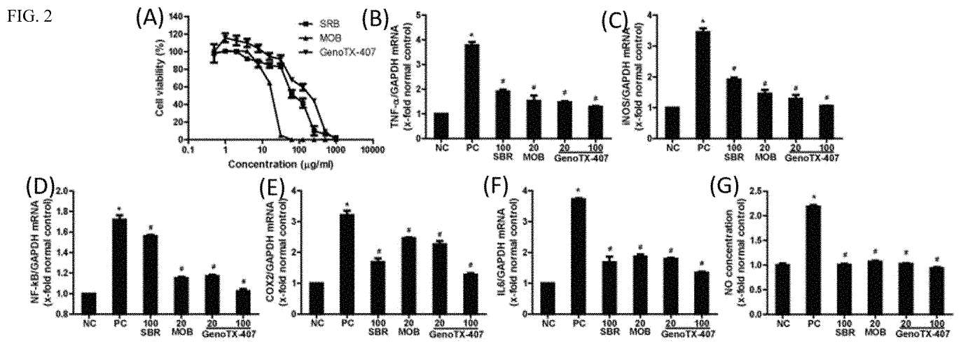

As shown in FIG. 2A, the low concentration (0 to 10 μg/mL) did not significantly affect cell survival. The concentrations required to inhibit the cell viability by 50% were about 95.46 and 15.89 μg/mL in Comparative Example 1 (SBR) and Comparative Example 2 (MOB), respectively. At concentrations of 250 and 31.3 μg/mL, RAW 264.7 growth was almost completely inhibited by SBR and MOB, respectively. The LD₅₀ of Present Example 1 (GenoTX-407) was 131.9 μg/mL.

[Experiment 3: Anti-Inflammatory Marker Test for Measuring Anti-Inflammatory Effect]

Quantitative Real-Time Polymerase Chain Reaction (qRT-PCR)

Total RNA was isolated from cells and brain tissue using TRIzol reagent (Invitrogen, Carlsbad, Calif., USA) according to the manufacturer's instructions. qRT-PCR was performed according to previously described methods. Glyceraldehyde 3-phosphate dehydrogenase (GAPDH) was used as an internal standard to normalize the expression of the target transcripts. The primer sets used to amplify tumor necrosis factor-alpha (TNF-α), inducible nitric oxide synthase (iNOS), nuclear factor-κB (NF-κB), prostaglandin-endoperoxide synthase 2 (COX2), and interleukin-6 (IL-6) are shown in Table 3. Triplicate data were analyzed by five independent assays using the comparative Ct method.

TABLE 3

| Gene | | Mouse Primer | |
|---|---|---|---|
| name | Accession No. | Forward (5’-3’) | Reverse (5’-3’) |
| COX2 | NM_011198 | GAACCTGCAGTTTGCTGTGG | ACTCTGTTGTGCTCCCGAAG |
| IL-6 | NM_031168.1 | TCCAGTTGCCTTCTTGGGAC | AGTCTCCTCTCCGGACTTGT |
| iNOS | NM_010927.3 | CTATGGCCGCTTTGATGTGC | TTGGGATGCTCCATGGTCAC |
| NF-κB | NM_008689 | CACTGCTCAGGTCCACTGTC | CTGTCACTATCCCGGAGTTCA |
| TNFα | NM_013693 | TACCTTGTTGCCTCCTCTT | GTCACCAAATCAGCGTTATTAAG |
| GAPDH | NM_008084 | CGTGCCGCCTGGAGAAACC | TGGAAGAGTGGGAGTTGCTGTTG |

### Sequences of the Primers Used in the Current Study

Nitric Oxide (NO) Production in RAW 264.7 Cells

The RAW 264.7 cells (1×10⁶ cells/mL) were seeded on a 24-well tissue culture plate and pre-incubated at 37° C. for 12 h to achieve stable attachment. Next, the wells were washed with phosphate-buffered saline (PBS), refreshed with media containing lipopolysaccharide (LPS, 1 μg/mL), Comparative Example 1 (SBR) (25, 50 and 100 μg/mL), Comparative Example 2 (MOB) (5, 10 and 20 μg/mL) and Present Example 1 (GenoTX-407) (20 and 100 μg/mL), and incubated for 24 h. Nitric oxide (NO) production was then monitored by measuring nitrite levels in the culture media using the Griess Reagent System (Promega Corporation, Madison, USA).

In order to analyze the anti-inflammatory effects of Present Example 1 (GenoTX-407), Comparative Example 1 (SBR) and Comparative Example 2 (MOB), the effects of qRT-PCR on the NF-κB pathway in RAW 264.7 cells were investigated.

Referring to the results in FIG. 2, the treatment with Present Example 1 (GenoTX-407) exhibited the greatest inhibitory effect on LPS-induced NF-κB upregulation. Simultaneously with the change of NF-κB, the expression of TNF-α and IL-6 was also significantly increased by LPS. This was sufficiently blocked by treatment with Comparative Example 1 (SBR), Comparative Example 2 (MOB) or Present Example 1 (GenoTX-407). According to the upregulation of NF-κB and pro-inflammatory cytokines, gene expression of the inflammatory enzymes, iNOS and COX2, increased after exposure to LPS. Referring to FIG. 2, treatment with Comparative Example 1 (SBR), Comparative Example 2 (MOB) and Present Example 1 (GenoTX-407) significantly suppressed the enzyme expression in a concentration-dependent manner. Similarly, LPS treatment significantly produced NO. Treatment of Comparative Example 1 (SBR), Comparative Example 2 (MOB) and Present Example 1 (GenoTX-407) together suppressed this upregulation.

[Experiment 4: Agar Disk Diffusion Assay]

Test Microorganisms and Growth Conditions

The strains used to evaluate the antibacterial effect of the extracts were Gram-positive bacteria (S. aureus ATCC 6538, S. epidermidis ATCC 12228, B. subtilis ATCC 33608 and P. acnes ATCC 9027), and Gram-negative bacteria (E. coli ATCC 8739, P. aeruginosa ATCC 9097), yeast (C. albicans ATCC 10231, S. cerevisiae ATCC 13007) and fungi (A. niger ATCC 16404, T. rubrum ATCC 62345). These were obtained from the Korean Culture Center of Microorganisms (KCCM, Seoul, Korea) or the Korean Collection for Type Cultures (KCTC, Jeongeup-si, Korea). E. coli, Bacillus subtilis and P. aeruginosa were cultured in nutrient agar (BD Difco, NJ, USA). Staphylococcus aureus (S. aureus) and Epidermidis (S. epidermidis) were cultured in Trypticase™ soy agar (BD Difco, USA). S. epidermidis was cultured on yeast mold (YM) agar (BD Difco, USA.) for 24 h at 37° C. after inoculation thereto. C. albicans and T. rubrum were cultured on YM agar and A. niger was cultured on potato dextrose agar (BD Difco, USA) for 3 to 5 days at 30° C. after inoculation thereto. P. acnes was cultured on reinforced clostridial agar (Oxoid Ltd., Hampshire, UK) for 2 to 3 days at 25° C. after inoculation thereto under anaerobic conditions in an anaerobic jar containing AnaeroGen sachets (Oxoid Ltd.) to maintain anaerobic conditions.

Antibacterial Test

Then, antimicrobial tests were carried out by the agar disk diffusion method using 100 μl of a suspension containing 10⁷ CFU/mL of bacteria and 10⁶ CFU/mL of yeast spread onto the specific agar plates, respectively. The disks (8 mm in diameter, ADVANTEC CO., LTD., Tokyo, Japan) were impregnated with 20 μl of the 100 mg/ml extracts (2 mg/disk) and placed on the inoculated agar. DMSO-loaded disks were used as negative controls. The inoculated plates were incubated at 37° C. for 24 h for bacterial strain and at 30° C. for 2 to 5 days for yeast and fungi. Antibacterial activity was evaluated by measuring the diameter of the clear zones around the disks. Antibacterial activity was expressed as the mean zone of inhibition diameters (mm) produced by the extract. Each assay in this experiment was repeated three times.

Minimum Inhibitory Concentration (MIC) and Minimum Bactericidal Concentration (MBC) Determination

The minimum inhibitory concentration (MIC) values were measured for the microbial strains which were sensitive to the extracts in the agar disk diffusion assay. Test microbial strains were incubated using broth media. The absorbances of the cultured suspensions were measured at 660 nm using a spectrophotometer and diluted to attain viable cell counts of 10⁶ CFU/mL for bacteria, and 10⁵ CFU/mL for yeast and fungi. The extracts were dissolved in DMSO, were first diluted to the highest stock concentration (100 mg/mL), and then serial two-fold dilutions were made in order to obtain a stock concentration range from 0.0031 to 50 mg/mL in 10 mL sterile test tubes containing DMSO. The MIC values of the extracts against bacterial strains or yeasts were determined based on a microwell dilution method with some modifications.

The 96-well plates were prepared by dispensing 185 μl of broth and 5 μl of the inoculum into each well. Then, 10 μlL of extract dissolved in DMSO (0.0031-100 mg/mL) was added into each well. The final volume in each well was 200 μl and the final concentration of the extracts was 0.153 to 5000 μg/mL. The contents of each well were thoroughly mixed with a multichannel pipette and the microplates were incubated at 37° C. for 24 h for bacterial strains and at 30° C. for 2 to 5 days for yeast and fungi. Microbial cell viability and MIC values were determined by observing the turbidity of the suspension after the incubation.

The minimum bactericidal concentration (MBC) of the contents were determined by taking a loopful of inoculum from each well of the microtiter plate with clear contents and streaking it onto the specific agar plates for bacterial and yeast species, respectively. The bacteria and yeast-streaked plates were incubated for the same incubation condition as previously. Then, the MBCs were determined as the lowest concentration of the extract that permits no growth of bacteria or yeast.

Specifically, using an agar disk diffusion and microdilution method for pathogenic microorganisms, in vitro tests were performed on the antimicrobial activity of Present Example 1 (GenoTX-407), Comparative Example 1 (SBR) to Comparative Example 2 (MOB). Table 4 below shows the antibacterial activity test results of Present Example 1 (GenoTX-407), Comparative Example 1 (SBR), and Comparative Example 2 (MOB) as determined by the disk diffusion assay.

TABLE 4
Inhibition diameter (mm)* around disk impregnated with
20 μL of each extract (2 mg/disk)

| | DMSO | SBR | MOB | GenoTX-407 |
|---|---|---|---|---|
| Escherichia coli | ND | ND | ND | 10.20 ± 0.131 |
| ATCC 8739 | | | | |
| Staphylococcus | ND | ND | 16.53 ± 0.361 | 18.90 ± 0.335 |
| aureus | | | | |
| ATCC 6538 | | | | |
| Candida albicans | ND | ND | 16.48 ± 0.055 | 19.18 ± 0.155 |
| ATCC 10231 | | | | |
| Propionibacterium | ND | ND | 30.54 ± 0.922 | 35.10 ± 0.676 |
| acnes | | | | |
| ATCC 9027 | | | | |
| Staphylococcus | ND | ND | 19.10 ± 0.259 | 24.04 ± 0.492 |
| epidermidis | | | | |
| ATCC 12226 | | | | |
| Pseudomonas | ND | ND | ND | 9.89 ± 0.083 |
| aeruginosa | | | | |
| ATCC 9097 | | | | |
| Bacillus subtilis | ND | ND | 20.70 ± 0.516 | 24.41 ± 0.492 |
| ATCC 33608 | | | | |
| Saccharomyces | ND | ND | 22.61 ± 1.266 | 28.12 ± 0.829 |
| cerevisiae | | | | |
| ATCC 13007 | | | | |
| Aspergillus ruger | ND | ND | 17.03 ± 0.871 | 18.92 ± 0.472 |
| ATCC 16404 | | | | |
| T. rubrum | ND | ND | 49.77 ± 0.240 | 50.47 ± 0.200 |
| ATCC 62345 | | | | |

Table 5 below shows the minimum inhibitory concentration (MIC) and minimum bactericidal concentration (MBC) (μg/ml) of Present Example 1 (GenoTX-407), Comparative Example 1 (SBR) and Comparative Example 2 (MOB).

TABLE 5

| Microbial | SBR | | MOB | | GenoTX-407 | |
|---|---|---|---|---|---|---|
| strains | MIC | MBC | MIC | MBC | MIC | MBC |
| Escherichia coli | 2500 | >5000 | 78.1 | 78.1 | 39.1 | 39.1 |
| ATCC 8739 | | | | | | |
| Staphylococcus aureus | —* | NA | 39.1 | 39.1 | 1.5 | 19.5 |
| ATCC 6538 | | | | | | |
| Candida albicans | — | NA | 9.77 | 9.77 | 2.44 | 2.44 |
| ATCC 10231 | | | | | | |
| propionibacterium acnes | — | NA | 2.44 | 9.77 | 2.44 | 9.77 |
| ATCC 9027 | | | | | | |
| Staphylococcus | — | NA | 4.88 | 4.88 | 4.88 | 4.88 |
| epidermidis | | | | | | |
| ATCC 12228 | | | | | | |
| Pseudomonas | — | NA | — | NA | 2500 | 2500 |
| aeruginosa | | | | | | |
| ATCC 9097 | | | | | | |
| Bacillus subtilus | — | NA | 9.77 | 19.5 | 9.77 | 19.5 |
| ATCC 33608 | | | | | | |
| Saccharomyces | — | NA | 9.77 | 19.5 | 9.77 | 19.5 |
| cerevisiae | | | | | | |
| ATCC 13007 | | | | | | |
| Aspergillus niger | — | NA | 78.1 | 78.1 | 78.1 | 78.1 |
| ATCC 16404 | | | | | | |
| T. rubum | — | NA | 4.88 | 4.88 | 4.88 | 4.88 |
| ATCC 62345 | | | | | | |

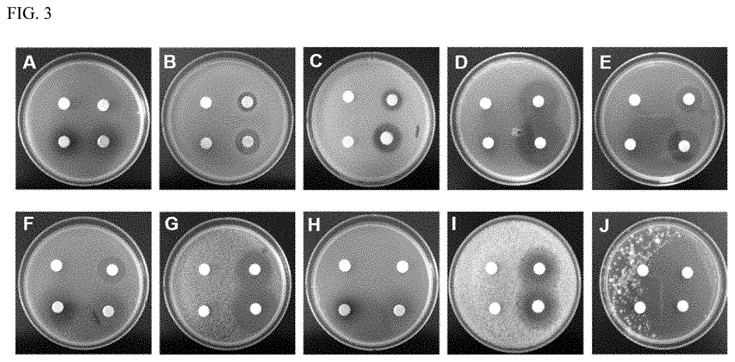

Referring to Table 4, Table 5, and FIG. 3, Present Example 1 (GenoTX-407) exhibited the highest antimicrobial activity against all of bacteria and fungi used in the present disclosure.

In the disc diffusion analysis, Comparative Example 1 (SBR) did not exhibit antimicrobial activity against any microorganisms. However, in the microdilution analysis, Comparative Example 1 (SBR) exhibited a MIC of 2500 μg/mL against E. coli. In general, Comparative Example 2 (MOB) was less effective than Present Example 1 (GenoTX-407) and did not affect the growth of Gram-negative organisms, E. coli and P. aeruginosa. However, treatment with Present Example 1 (GenoTX-407) was only the treatment exhibiting antibacterial activity against E. coli (10.20±0.131 mm) and P. aeruginosa (9.89±0.083 mm). From the MBC value, it was identified that Present Example 1 (GenoTX-407) also exhibited a bactericidal effect. Among Gram-positive bacteria, Propionibacterium acnes (P. acnes) was the most sensitive strain to the treatment with Comparative Example 2 (MOB) and Present Example 1 (GenoTX-407). The zones of inhibition thereof were 30.54±0.922 and 35.10±0.676 mm, respectively. In terms of MBC values, Comparative Example 2 (MOB) and Present Example 1 (GenoTX-407) exhibited a bacteriostatic effect on Propionibacterium acnes (P. acnes), whereas Comparative Example 2 (MOB) and Present Example 1 had the inhibition zones of 16.53±0.361 and 18.90±0.335 mm respectively against S. aureus which was the most resistant thereto. Moreover, S. epidermidis and B. substilis were less sensitive to the treatment with Comparative Example 2 (MOB) and Present Example 1 (GenoTX-407), respectively. Comparative Example 2 (MOB) and Present Example 1 (GenoTX-407) exhibited bactericidal effects, and thus exhibited significant bactericidal activity against yeasts, Candida albicans (C. albicans) (16.48±0.055 and 19.18±0.155 mm, respectively), and Saccharomyces cerevisiae (S. cerevisiae) (22.61±1.266 and 28.12±0.829 mm, respectively).

[Experiment 5: Western Blot Analysis]

HeLa cells were seeded on a 6-well tissue culture plate and pre-incubated at 37° C. for 12 h to achieve stable attachment. Next, the wells were washed with PBS, refreshed with media containing Comparative Example 1 (SBR) (100 μg/mL), Comparative Example 2 (MOB) (20 μg/mL), and Present Example 1 (GenoTX-407) (20, 50 and 100 μg/mL), respectively, and incubated for 24 h. The HeLa cells were homogenized in 10 volumes of radioimmunoprecipitation assay (RIPA) buffer (Sigma-Aldrich, MO, USA) containing protease inhibitors (Sigma-Aldrich) and phosphatase inhibitors (Sigma-Aldrich). Western blot analysis was conducted according to previously described methods. The membranes were then immunoblotted with primary antibodies, followed by incubation with horseradish peroxidase-conjugated anti-rabbit antibodies (Cell signaling Technology, Danvers, Mass., USA). The antibodies used in the present disclosure are Nrf2 (Abeam, Cambridge, UK), HO-1 (Abeam), SOD1 (Abeam), and CAT (Abeam). The band densities were measured using ImageJ software (National Institutes of Health, NIH, Maryland, USA) and normalized to the density of actin.

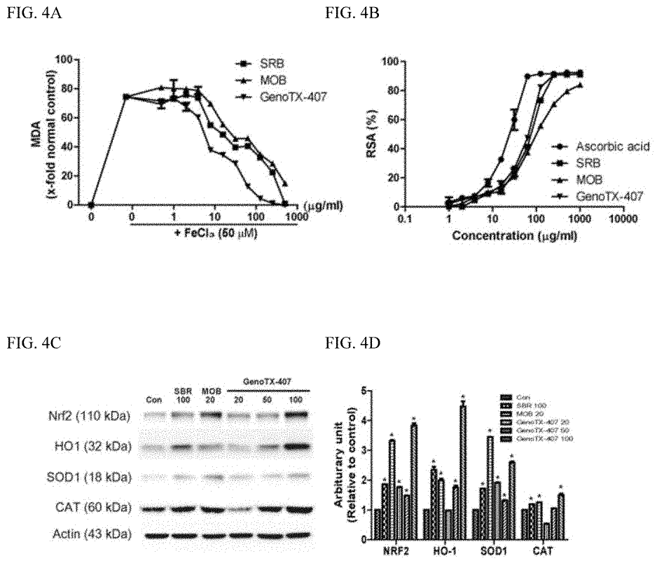

FIGS. 4A through 4D show lipid peroxidation and DPPH (1,1-diphenyl-2-picrylhydrazyl) analysis result which were obtained by measuring the antioxidant activity of Comparative Example 1 (SBR), Comparative Example 2 (MOB) and Present Example 1 (GenoTX-407). FIG. 4A shows the inhibitory activity against FeCl₃-induced lipid peroxidation (TBARS generation). FIG. 4B shows 1,1-diphenyl-2-picrylhydrazyl (DPPH) radical scavenging activity in 30 minutes. FIG. 4C shows Western blot results of Nrf2, HO-1, SOD1 and CAT proteins. FIG. 4D shows the measurement of the band density standardized with GAPDH.

As shown in the upper left graph of FIG. 4, after exposure to FeCl₃ (50 μM), TBARS (thiobarbituric acid reactive substances) level increased (74%). However, treatment with various concentrations (0 to 500 μg/mL) of Comparative Example 1 (SBR), Comparative Example 2 (MOB) and Present Example 1 (GenoTX-407) significantly decreased lipid peroxidation depending on the concentration. Similarly, in DPPH assay, these treatments significantly increased radical scavenging activity in a dose-dependent manner. Interestingly, Present Example 1 (GenoTX-407) (EC50, 7.706 μg/mL and 26.41 μg/mL) exhibited greater ability to lower lipid peroxidation and DPPH, compared to those of Comparative Example 1 (SBR) (107.7 μg/mL and 38.30 μg/mL) and Comparative Example 2 (MOB) (62.41 μg/mL and 72.45 μg/ml). However, in DPPH analysis, all treatments with Comparative Example 1 (SBR), Comparative Example 2 (MOB) and Present Example 1 (GenoTX-407) were less potent than treatment with ascorbic acid (14.38 μg/mL) as the positive control was. In Western blot analysis, the expression of proteins of the antioxidative enzymes SOD1 and CAT, as well as Nrf2 and HO-1, was markedly upregulated by treatments with Comparative Example 1 (SBR), Comparative Example 2 (MOB) and Present Example 1 (GenoTX-407). Notably, the Present Example 1 (GenoTX-407) (100 μg/mL) resulted in higher upregulation of these enzymes than Comparative Example 1 (SBR) (100 μg/mL) and Comparative Example 2 (MOB) (20 μg/mL).

Statistical Analysis

Statistical comparisons between the groups were performed using one-way analysis of variance (ANOVA) followed by the Tukey's multiple comparison test. All analyses were conducted while using the Statistical Package for Social Sciences software, version 12.0 (SPSS Inc., IL, USA). Statistical significance was assessed at p<0.05. All data were expressed as mean±standard deviation (SD).

In summary, the present inventors proved based on the above experiments that Present Example 1 (GenoTX-407), Comparative Example 1 (SBR), and Comparative Example 2 (MOB) exhibited antioxidant, antibacterial and anti-inflammatory effects in a concentration-dependent manner. In this connection, Comparative Example 1 (SBR) has stronger antioxidant and anti-inflammatory activity than Comparative Example 2 (MOB) has. In contrast, Comparative Example 2 (MOB) has a stronger antibacterial activity than Comparative Example 1 (SBR) has.

However, Present Example 1 (GenoTX-407) containing the mixture of SBR and MOB extracts at a specific ratio based on parts by weight exhibited synergistic effects exceeding a simple sum of effect of single Comparative Example 1 and effect of single Comparative Example 2 in the antioxidant and anti-inflammatory activity tests. In addition, it is identified that Present Example 1 (GenoTX-407) exhibits the highest antibacterial effect against all bacteria and fungi used in the present disclosure, compared with Comparative Example 2 (MOB).

In addition, it is well known that baicalin is a predominant flavonoid isolated from SBR with a defined chemical constitution. In the present disclosure, we tried to find the best conditions for extraction using baicalin. Although there are several factors, other studies reported that extraction time is the major determining factor in the optimization of an extraction process. Therefore, in the present disclosure, the effects of two experimental factors such as ethanol concentration and extraction time on baicalin yield from SBR were studied by HPLC analysis. As a result, we verified the stability of the optimized extraction process by determining the optimum conditions such as ethanol concentration, 0%, and extraction time, 2 h, which confirmed that the optimized extraction process is stable and precise. Similarly, magnolol and honokiol are major components isolated from MOB and the optimized conditions are an ethanol concentration of 50% and an extraction time of 2 h.

It is identified that baicalin has various pharmacological activities, including antioxidant, antiviral, anti-inflammatory and antiproliferative activities. In the present disclosure, baicalin (SBR) also had potent antioxidant and anti-inflammatory effects as determined in the DPPH assay and LPS-stimulated RAW 264.7 cells. Baicalin (SBR) not only inhibited LPS-induced inflammatory responses and NF-κB activity, but also enhanced the expression of Nrf2, HO-1, and antioxidant enzymes. These findings strongly support that baicalin (SBR) treatment offered protection against oxidative stress and decreased NO production and inflammation by activation of the Nrf2-HO-1 pathway. Similarly, it was reported that honokiol and magnolol have strong antioxidative, anti-inflammatory, antitumor, and antimicrobial properties mediated by several modes of action. In the present disclosure, we confirmed the antioxidant and anti-inflammatory effects of honokiol and magnolol (MOB) exerted through the Nrf2/HO-1 pathway.

Interestingly, Comparative Example 1 (SBR) treatment was less effective against bacteria than Comparative Example 2 (MOB) treatment was. Comparative Example 2 (MOB) exhibited activity against some bacteria. However, Present Example 1 (GenoTX-407) exhibited the highest antibacterial activity against all bacteria and fungi investigated in the present disclosure. Further, the co-treatment with baicalin increased the growth-inhibitory activity of antibiotics.

Baicalin interferes with cell wall integrity through direct binding to peptidoglycan, and it exerts synergistic antimicrobial activity with tetracycline and β-lactams. Also, it was reported that baicalin restored the inhibition of TetK-mediated tetracycline efflux. Similarly, magnolol and honokiol in MOB have been known to show high antimicrobial activity against several microorganisms such as fungi, Propionibacterium species, and S. aureus. They have shown synergistic effects with several representative antibiotics such as oxacillin, ampicillin, chloramphenicol, tetracycline, and cefoxitin.

Therefore, in the present disclosure, it was identified that when the SBR and MOB extracts were combined with each other (GenoTX-407) in a specific ratio based on parts by weight, the most potent antibacterial activity was achieved.

It was reported that β-glucans play an important role in protection against vulvovaginal candidiasis associated with C. albicans, via its antioxidative, anti-inflammatory, and antimicrobial properties. Similarly, the Present Example 1 (GenoTX-407) exhibited potent antioxidative and anti-inflammatory activity through the Nrf2/HO-1 signaling pathway. Particularly, the Present Example 1 (GenoTX-407) inhibited the growth of C. albicans. This suggests that the Present Example 1 (GenoTX-407) may be a useful antimicrobial drug against vaginitis.

Taken together, the results obtained in the present disclosure suggest that GenoTX-407 as the combination between the SBR and MOB extracts in a specific ratio based on parts by weight may be a useful candidate substance to create synergistic effects with antibiotics and may lead to the development of a novel combination drug for the treatment of vulvovaginal candidiasis.

From the foregoing, it will be appreciated that various examples of the present disclosure have been described herein for purposes of illustration, and that various modifications may be made without departing from the scope and spirit of the present disclosure. Accordingly, the various examples disclosed herein are not intended to be limiting, with the true scope and spirit being indicated by the following claims.

What is claimed is:

1. An antioxidant, antibacterial or anti-inflammatory pharmaceutical composition containing a Scutellaria baicalensis extract and a Magnolia officinalis extract as active ingredients.

2. The composition of claim 1, wherein the composition contains the Scutellaria baicalensis extract and the Magnolia officinalis extract in a ratio of 4 to 6:1 based on parts by weight.

3. The composition of claim 1, wherein the Scutellaria baicalensis extract is extracted using water for 1 to 3 hours at 70 to 90° C.

4. The composition of claim 1, wherein the Magnolia officinalis extract is extracted using 50% ethanol for 1 to 3 hours at 50 to 70° C.

5. The composition of claim 1, wherein the Scutellaria baicalensis extract is extracted using Scutellaria baicalensis and a solvent in a ratio of 1:7 to 9 based on parts by weight, and the Magnolia officinalis extract is extracted using Magnolia officinalis and a solvent in a ratio of 1:7 to 9 based on parts by weight.

6. The composition of claim 1, wherein each of the Scutellaria baicalensis extract and the Magnolia officinalis extract is a fraction extract obtained by fractionation with at least one fractionation solvent selected from a group consisting of butanol, acetone, phosphoric acid, hexane, ethanol, and water.

7. The composition of claim 1, wherein the composition exhibits an antibacterial effect against at least one selected from a group consisting of E. coli, P. aeruginosa, P. acnes, S. aureus, S. epidermidis, B. substilis, C. albicans, and S. cerevisiae.

8. The composition of claim 1, wherein the composition has a prophylactic, therapeutic or ameliorating effect of vulvovaginal candidiasis.
